SEQUENCE LISTING (23 sequences)

SEQ ID NO: 1 - DNA, 373 bp, West Nile virus
cggaaagttgataacagattggtgctgcaggagctgcaccttaccaccactgcgctaccaaactgacagcggctgttggtatggtatggagatcagaccacagagacatgatgaaaagaccctcgtgcagtcacaagtgaatgcttataatgctgatatgattgacccttttcagttgggccttctggtcgtgttcttggccacccaggaggtccttcgcaagaggtggacagccaagatcagcatgccagctatactgattgctctgctagtcctggtgtttgggggcattacttacactgatgtgttacgctatgtcatcttggtgggggcagctttcgcagaatctaattcgggaggagacgtggtacac

SEQ ID NO: 2 - DNA, 400 bp, West Nile virus
cactgagagtgggaagctcatcacagactggtgctgcagaagttgcaccctccctccactgcgcttccagactgagaatggctgttggtatggaatggaaattcgacctacgcggcacgacgaaaagaccctcgtgcaatcgagagtgaatgcatacaacgccgacatgattgatccttttcagttgggccttctggtcgtgttcttggctacccaggaggtccttcgcaagaggtggacggccaagatcagcattccagctatcatgcttgcactcctagtcctagtgtttgggggtattacgtacactgatgtcctgcgatatgtcattctcgtcggcgccgcgtttgctgaagcaaactcaggaggagacgtcgtgcacttggcacttatggctaca

SEQ ID NO: 3 - DNA, 400 bp, West Nile virus
cactcgcaccaccacagagagcggaaagttgataacagattggtgctgcaggagctgcaccttaccaccactgcgctaccaaactgacagcggctgttggtatggtatggagatcagaccacagagacatgatgaaaagaccctcgtgcagtcacaagtgaatgcttacaatgctgatatgattgacccttttcagttgggccttctggtcgtgttcttggccacccaggaggtccttcgcaagaggtggacagccaagatcagcatgccagctatactgattgctctgttagtcctggtgtttgggggcattacttacactgatgtgttacgctatgtcattttggtgggggcagcttttgcagaatctaattcgggaggagacgtggtacacttggcg

SEQ ID NO: 4 - DNA, 400 bp, West Nile virus
cgcgcgcaccaccacagagagtgggaagttgatcacggattggtgctgcagaagctgcacgctccccccactacggtatcaaactgatagtggatgttggtatggaatggaaatcagacctttgaagcatgatgagaagacgttggttcaatctagggtgagcgcctacaaatctgatatgattgatccttttcagctgggccttctggtagtgttcttggccacccaggaggtcctccgcaagaggtggacagccaagatcagcattcctgctattctggtcgctcttgcagtcctagtgcttgggggcatcacttacactgatgttctgagatacatcattcttgtgggtgcggcctttatggaagccaactcaggtggagatgtggtgcatcttgct

SEQ ID NO: 5 - DNA, 20 bp, Artificial Sequence
  Synthetic oligonucleotide
tgttggtatggtatggagat

SEQ ID NO: 6 - DNA, 20 bp, Artificial Sequence
  Sythetic oligonucleotide
tgttggtatggaatggaaat

SEQ ID NO: 7 - DNA, 18 bp, Artificial Sequence
  Sythetic oligonucleotide
agtgtaagtaatgccccc

SEQ ID NO: 8 - DNA, 18 bp, Artificial Sequence
  Sythetic oligonucleotide
agtgtacgtaataccccc

SEQ ID NO: 9 - DNA, 27 bp, Artificial Sequence
  Sythetic oligonucleotide
atgattgacccuuuucagttgggcctt

SEQ ID NO: 10 - protein, 224 aa, Pyrococcus furiosus
MKIGGIDEAGRGPAIGPLVVATVVVDEKNIEKLRNIGVKDSKQLTPHERKNLFSQITSIADDYKIVIVSPEEIDNRSGTMNELEVEKFALALNSLQIKPALIYADAADVDANRFASLIERRLNYKAKIIAEHKADAKYPVVSAASILAKVVRDEEIEKLKKQYGDFGSGYPSDPKTKKWLEEYYKKHNSFPPIVRRTWETVRKIEESIKAKKSQLTLDKFFKKP

SEQ ID NO: 11 - protein, 16 aa, Artificial Sequence
  Synthetic polypeptide
GIDEAGRGPAIGPLVV

SEQ ID NO: 12 - protein, 12 aa, Artificial Sequence
  Synthetic polypeptide
LRNIGVKDSKQL

SEQ ID NO: 13 - protein, 19 aa, Artificial Sequence
  Synthetic polypeptide
HKADAKYPVVSAASILAKV

SEQ ID NO: 14 - protein, 16 aa, Artificial Sequence
  Synthetic polypeptide
KLKKQYGDFGSGYPSD

SEQ ID NO: 15 - protein, 166 aa, Thermus thermophilus
MNPSPRKRVALFTDGACLGNPGPGGWAALLRFHAHEKLLSGGEACTTNNRMELKAAIEGLKALKEPCEVDLYTDSHYLKKAFTEGWLEGWRKRGWRTAEGKPVKNRDLWEALLLAMAPHRVRFHFVKGHTGHPENERVDREARRQAQSQAKTPCPPRAPTLFHEEA

SEQ ID NO: 16 - protein, 25 aa, Artificial Sequence
  Synthetic polypeptide
KXVXLFTDGXCXGNPGXGGXALLRY

SEQ ID NO: 17 - protein, 8 aa, Artificial Sequence
  Synthetic polypeptide
TTNNRMEL

SEQ ID NO: 18 - protein, 5 aa, Artificial Sequence
  Synthetic polypeptide
KPVKN

SEQ ID NO: 19 - protein, 12 aa, Artificial Sequence
  Synthetic polypeptide
FVKGHXGHXENE

SEQ ID NO: 20 - DNA, 228 bp, West Nile virus
tgttggtatggtatggagatcagaccacagagacatgatgaaaagaccctcgtgcagtcacaagtgaatgcttataatgctgatatgattgacccttttcagttgggccttctggtcgtgttcttggccacccaggaggtccttcgcaagaggtggacagccaagatcagcatgccagctatactgattgctctgctagtcctggtgtttgggggcattacttacact

SEQ ID NO: 21 - DNA, 228 bp, West Nile virus
tgttggtatggtatggagatcagaccacagagacatgatgaaaagaccctcgtgcagtcacaagtgaatgcttacaatgctgatatgattgacccttttcagttgggccttctggtcgtgttcttggccacccaggaggtccttcgcaagaggtggacagccaagatcagcatgccagctatactgattgctctgttagtcctggtgtttgggggcattacttacact

SEQ ID NO: 22 - DNA, 228 bp, West Nile virus
tgttggtatggaatggaaattcgacctacgcggcacgacgaaaagaccctcgtgcaatcgagagtgaatgcatacaacgccgacatgattgatccttttcagttgggccttctggtcgtgttcttggctacccaggaggtccttcgcaagaggtggacggccaagatcagcattccagctatcatgcttgcactcctagtcctagtgtttgggggtattacgtacact

SEQ ID NO: 23 - DNA, 228 bp, West Nile virus
tgttggtatggaatggaaatcagacctttgaagcatgatgagaagacgttggttcaatctagggtgagcgcctacaaatctgatatgattgatccttttcagctgggccttctggtagtgttcttggccacccaggaggtcctccgcaagaggtggacagccaagatcagcattcctgctattctggtcgctcttgcagtcctagtgcttgggggcatcacttacact

(19) United States
(12) Patent Application Publication
(10) Pub. No.: US 2013/0029316 A1
(43) Pub. Date: Jan. 31, 2013

(54) METHOD FOR REAL-TIME DETECTION OF WEST NILE VIRUS USING A CLEAVABLE CHIMERIC PROBE

(71) Applicant: John HARVEY, Elkridge, MD (US)

(72) Inventor: John HARVEY, Elkridge, MD (US)

(73) Assignee: SAMSUNG TECHWIN CO., LTD., Changwon-city (KR)

(21) Appl. No.: 13/192,892

(22) Filed: Jul. 28, 2011

(51) Int. Cl. C12Q 1/70

(52) U.S. Cl. 435/5

(57) ABSTRACT

A method is described for the real-time detection of West Nile Virus in samples taken from humans or potential carriers of the virus such as mosquitoes or birds. Real-time detection of West Nile Virus is performed in a PCR reaction using gene specific primers and a cleavable chimeric fluorescent probe. The method is amenable to medium and high throughput analysis.

### FIELD

The disclosure describes a method and a test kit for the real-time detection of West Nile Virus.

### BACKGROUND

The West Nile Virus (WNV) is a single-stranded RNA virus of the family Flaviviridae, genus Flavivirus. WNV is the etiologic agent of the mosquito-borne West Nile Virus disease, particularly the potentially fatal West Nile encephalitis, in humans and other mammals. Believed to be transported between and within countries through infected migratory birds, the virus can be transmitted through mosquitoes to a variety of hosts including birds, humans, horses, dogs, cats, bats, chipmunks, skunks, squirrels, domestic vamide rabbits, crocodiles and alligators.

Initially isolated in 1937, WNV is now recognized as one of the most widely distributed flaviviruses, endemic in Africa, Europe, the Middle East, and parts of Asia. Since 1999, the virus has been recognized in North America by causing an epizootic among birds and horses and an epidemic of meningitis and encephalitis in humans. Currently, no specific vaccine or therapy has been approved for human use.

According to the Centers for Disease Control and Prevention, about one in 150 people infected with WNV will develop potentially life threatening neuro-invasive disease termed West Nile meningitis or encephalitis. The severe symptoms can include high fever, headache, neck stiffness, stupor, disorientation, coma, tremors, convulsions, muscle weakness, vision loss, numbness and paralysis. These symptoms may last several weeks, and neurological effects may be permanent and can result in death. Up to 20% of WNV infected patient develop the more mild febrile syndrome, termed West Nile Fever, characterized by flu-like symptoms that can last from a few days to several weeks. An estimated 80% of WNV infections are asymptomatic.

Early detection of WNV in transmitting hosts such as migratory birds and mosquitos is key to controlling the spread of an outbreak and reducing morbidity associated with neuro-invasive disease.

One of the most widely used techniques to detect viral gene expression exploits first-strand cDNA of mRNA sequence(s) as a template for PCR amplification. The ability to measure the kinetics of a PCR reaction in combination with reverse transcriptase-PCR techniques promises to facilitate the accurate and precise measurement of viral target RNA sequences with the requisite level of sensitivity.

In particular, fluorescent dual-labeled hybridization probe technologies, such as the “CATACLEAVE™ endonuclease assay (described in detail in U.S. Pat. No. 5,763,181; see FIG. 1), permit the detection of reverse transcriptase-PCR amplification in real time. Detection of target sequences is achieved by including a CATACLEAVE™ probe in the amplification reaction together with RNase H. The CATACLEAVE™ probe, which is complementary to a target sequence within the PCR amplification product, has a chimeric structure comprising an RNA sequence and a DNA sequence, and is flanked at its 5′ and 3′ ends by a detectable marker, for example Förster Resonance Energy Transfer (FRET) pair labeled DNA sequences. The proximity of the FRET pair's fluorescent label to the quencher precludes fluorescence of the intact probe. Upon annealing of the probe to the nucleic acid target a RNA:DNA duplex is generated that can be cleaved by RNase H present in the reaction mixture. Cleavage within the RNA portion of the annealed probe results in the separation of the fluorescent label from the quencher and a subsequent emission of fluorescence.

### SUMMARY

Methods and kits are described for the rapid detection of West Nile Virus in humans as well as potential carriers such birds and mosquitoes. The procedure promises to facilitate the high throughput detection of WNV in a cost effective and reliable manner.

In one embodiment, there is disclosed a method for the real-time detection of West Nile Virus (WNV) in a sample, comprising the steps of (1) providing a sample to be tested for the presence of a WNV target nucleic acid sequence, (2) providing a pair of forward and reverse amplification primers, wherein the primer pair anneals to a WNV homology region of SEQ ID NO: 1, 2, 3 or 4 comprising the WNV target nucleic acid sequence, (3) providing a probe comprising a detectable label and DNA and RNA nucleic acid sequences, wherein the probe's RNA nucleic acid sequences are entirely complementary to a selected region of a cDNA of the target WNV and the probe's DNA nucleic acid sequences are substantially complementary to WNV cDNA sequences adjacent to the selected region of the target DNA sequence, (4) reverse transcribing the WNV target RNA in the presence of a reverse transcriptase activity and the reverse amplification primer to produce a target WNV cDNA sequence, (5) amplifying an PCR fragment between the forward and reverse amplification primers in the presence of the WNV target cDNA sequence, an amplifying polymerase activity, an amplification buffer; an RNAse H activity and the probe under conditions where the RNA sequences within the probe can form a RNA:DNA heteroduplex with complimentary sequences in the PCR fragment; and (6) detecting a real-time increase in the emission of a signal from the label on the probe, wherein the increase in signal indicates the presence of the WNV target nucleic acid sequences in the sample.

In another embodiment, there is disclosed a method for the real-time detection of West Nile Virus (WNV) in a sample, comprising the steps of (1) providing a sample to be tested for the presence of a WNV target nucleic acid sequence, (2) providing a pair of forward and reverse amplification primers that can anneal to the WNV target nucleic acid sequence, wherein the forward amplification primer can be the primer of SEQ ID NO: 5 or 6 and the reverse amplification primer can be the primer of SEQ ID NO: 7 or 8, (3) providing a probe comprising a detectable label and DNA and RNA nucleic acid sequences, wherein the probe's RNA nucleic acid sequences are entirely complementary to a selected region of a cDNA of the target WNV and the probe's DNA nucleic acid sequences are substantially complementary to WNV cDNA sequences adjacent to the selected region of the target DNA sequence, (4) reverse transcribing the WNV target RNA in the presence of a reverse transcriptase activity and the reverse amplification primer to produce a target WNV cDNA sequence, (5) amplifying an PCR fragment between the forward and reverse amplification primers in the presence of the WNV target cDNA sequence, an amplifying polymerase activity, an amplification buffer; an RNAse H activity and the probe under conditions where the RNA sequences within the probe can form a RNA:DNA heteroduplex with complimentary sequences in the PCR fragment; and (6) detecting a real-time increase in the emission of a signal from the label on the probe, wherein the increase in signal indicates the presence of the WNV target nucleic acid sequences in the sample.

In one aspect, the real-time increase in the emission of the signal from the label on the probe results from the RNAse H cleavage of the probe's RNA sequences in the RNA:DNA heteroduplex. In one embodiment, the real-time increase in the emission of a signal from the label on the probe can detect 100 copies of WNV lineage 1, 10 copies of WNV lineage 1A, 10 copies of WNV lineage 2 and 100 copies of WNV lineage 3.

In another embodiment, there is disclosed a kit for the real-time detection of West Nile Virus (WNV) in a sample, comprising (1) a reverse transcriptase activity for the reverse transcription of a target West Nile Virus (WNV) RNA sequence to produce a target cDNA sequence, (2) a pair of forward and reverse amplification primers that can anneal to the WNV target nucleic acid sequence, wherein the forward amplification primer can be the primer of SEQ ID NO: 5 or 6 and the reverse amplification primer can be the primer of SEQ ID NO: 7 or 8, (3) an amplifying activity for the PCR amplification of the target WNV cDNA sequence between the pair of amplification primers to produce a West Nile Virus (WNV) PCR fragment, a probe comprising a detectable label and DNA and RNA nucleic acid sequences, wherein the probe's RNA nucleic acid sequences are entirely complementary to a selected region of the target WNV cDNA and the probe's DNA nucleic acid sequences are substantially complementary to WNV cDNA sequences adjacent to the selected region of the target DNA sequence, and (4) an RNAse H activity.

The kit can have positive, internal, and negative controls and may also include an amplifying polymerase activity.

The amplifying polymerase activity can be an activity of a thermostable DNA polymerase. The RNAse H activity can be the activity of a thermostable RNAse H or a hot start RNAse H activity.

The DNA and RNA sequences of the probe may be covalently linked and the detectable label on the probe can be a fluorescent label such as a FRET pair. The PCR fragment or probe can be linked to a solid support.

The probe can have the nucleic acid sequence of SEQ ID NO: 9.

The previously described embodiments have many advantages, including the ability to detect West Nile Virus nucleic acid sequences in a sample in real-time. The detection method is fast, accurate and suitable for high throughput applications. Convenient, user-friendly and reliable diagnostic kits are also described for the detection of West Nile Virus infection in humans as well as potential carriers of the virus.

### BRIEF DESCRIPTION OF THE DRAWINGS

FIG. 1 is a schematic representation of CataCleave™ probe technology.

FIG. 2 is a schematic representation of real-time CataCleave™ probe detection of PCR amplification products.

FIG. 3 is the output of a real-time CataCleave™ PCR reaction to detect West Nile Virus lineage 1.

FIG. 4 is the output of a real-time CataCleave™ PCR reaction to detect West Nile Virus lineage 1A.

FIG. 5 is the output of a real-time CataCleave™ PCR reaction to detect West Nile Virus lineage 2.

FIG. 6 is the output of a real-time PCR reaction to detect West Nile Virus lineage 3.

FIG. 7 is the sequence alignment of four West Nile Virus isolates representing lineages 1, 1A, 2, and 3.

### DETAILED DESCRIPTION OF THE INVENTION

The practice of the embodiments described herein employs, unless otherwise indicated, conventional molecular biological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements; Sambrook, et al., Molecular Cloning: A Laboratory Manual, 2nd Edition, Cold Spring Harbor, N.Y. (1989).

Unless defined otherwise, all technical and scientific terms used herein have the same meaning as is commonly understood by one of skill in the art. The specification also provides definitions of terms to help interpret the disclosure and claims of this application. In the event a definition is not consistent with definitions elsewhere, the definition set forth in this application will control.

As used herein, the term “nucleic acid” refers to an oligonucleotide or polynucleotide, wherein said oligonucleotide or polynucleotide may be modified or may comprise modified bases. Oligonucleotides are single-stranded polymers of nucleotides comprising from 2 to 60 nucleotides. Polynucleotides are polymers of nucleotides comprising two or more nucleotides. Polynucleotides may be either double-stranded DNAs, including annealed oligonucleotides wherein the second strand is an oligonucleotide with the reverse complement sequence of the first oligonucleotide, single-stranded nucleic acid polymers comprising deoxythymidine, single-stranded RNAs, double stranded RNAs or RNA/DNA heteroduplexes. Nucleic acids include, but are not limited to, genomic DNA, cDNA, hnRNA, snRNA, microRNA, RNAi, mRNA, rRNA, tRNA, or fragmented nucleic acid. Nucleic acids may be composed of a single type of sugar moiety, e.g., as in the case of RNA and DNA, or mixtures of different sugar moieties, e.g., as in the case of RNA/DNA chimeras.

As used herein, the term “oligonucleotide” is used sometimes interchangeably with “primer” or “polynucleotide.” The term “primer” refers to an oligonucleotide that acts as a point of initiation of DNA synthesis in a PCR reaction. A primer is usually about 15 to about 35 nucleotides in length and hybridizes to a region complementary to the target sequence. Oligonucleotides may be synthesized and prepared by any suitable methods (such as chemical synthesis), which are known in the art. Oligonucleotides may also be conveniently available through commercial sources.

A “target cDNA or “target RNA”” or “target nucleic acid,” or “target nucleic acid sequence” refers to a nucleic acid that is targeted by DNA amplification. A target nucleic acid sequence, which can be either RNA or cDNA, serves as a template for amplification in a PCR reaction or reverse transcriptase-PCR reaction. Target nucleic acid sequences may include both naturally occurring and synthetic molecules.

The terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. In certain embodiments, base-stacking and hydrophobic interactions may also contribute to duplex stability.

### Selection of West Nile Virus Target Sequences

To select WNV target nucleic acid sequences for real-time PCR detection, the complete genome sequences of different WNV virus lineages are first aligned and examined for regions of homology. Candidate primers pair annealing to a selected homology regions are then screened for the formation of primer dimers.

As used herein, a “WNV homology region” refers to a nucleic acid sequences with at least 70%, 75%, 80%, 85%, 90%, 95%, 99% or 100% sequence identity with the nucleic acid sequences of SEQ ID NO: 1, 2, 3 or 4.

“Percentage of sequence identity” is determined by comparing two optimally aligned sequences over a comparison window. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity.

The determination of percent identity between any two sequences can be accomplished using a mathematical algorithm. Preferred, non-limiting examples of such mathematical algorithms are the algorithm of Myers and Miller, CABIOS, 4:11 (1988); the local homology algorithm of Smith et al., Adv. Appl. Math., 2:482 (1981); the homology alignment algorithm of Needleman and Wunsch, JMB, 48:443 (1970); the search-for-similarity-method of Pearson and Lipman, Proc. Natl. Acad. Sci. USA, 85:2444 (1988); the algorithm of Karlin and Altschul, Proc. Natl. Acad. Sci. USA, 87:2264 (1990), modified as in Karlin and Altschul, Proc. Natl. Acad. Sci. USA, 90:5873 (1993).

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

The CLUSTAL program is well described by Higgins et al., Gene, 73:237 (1988); Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994). The ALIGN program is based on the algorithm of Myers and Miller, supra. The BLAST programs of Altschul et al., JMB, 215:403 (1990); Nucl. Acids Res., 25:3389 (1990), are based on the algorithm of Karlin and Altschul supra.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

A “primer dimer” is a potential by-product in PCR, that consists of primer molecules that have partially hybridized to each other because of strings of complementary bases in the primers. As a result, the DNA polymerase amplifies the primer dimer, leading to competition for PCR reagents, thus potentially inhibiting amplification of the DNA sequence targeted for PCR amplification. In real-time PCR, primer dimers may interfere with accurate quantification by reducing sensitivity.

Candidate primer sequences for the detection of WNV minigene nucleic acid sequences are screened for the formation of primer-dimers. PCR reactions are performed using pairs of forward and reverse primers in the presence of Sybr Green I. The fluorescence emission intensity of this dye increases when it becomes intercalated into duplex DNA and therefore can serve as a non-specific probe in nucleic acid amplification reactions. The reactions are performed in a suitable reaction buffer containing 40 nM of forward and reverse primer, thermostable DNA polymerase, and Sybr Green I. Exemplary temperature cycling conditions were 95° C. for 5 minutes, followed by 40 cycles of 95° C. for 15 seconds, 55° C. for 15 seconds, and 72° C. for 30 seconds. Real-time data were collected during the 72° C. step. An increase in Sybr Green I fluorescence emission can be detected in real-time using a suitable instrument, such as the Applied Biosystems 7500 Fast Real-Time PCR System or the Biorad CFX96 real-time PCR thermocycler. Primer-dimer formation leads to a characteristic sigmoidal shaped emission profile similar to that seen in the presence of primer-specific template DNA.

A person of skill in the art would know how to design PCR primers flanking a West Nile Virus sequence of interest. Synthesized oligos can be between 20 and 26 base pairs in length with a melting temperature, T_{M} of about 55° C.

### Nucleic Acid Template Preparation

The sample can be a purified nucleic acid template (e.g., viral mRNA, viral RNA, total RNA, and mixtures thereof). In other embodiments, the sample may include a lysate of cultured cells and animal or human blood, serum, tissues or cerebrospinal fluid, but is not limited thereto.

Mosquitoes suspected of being infected with WNV can be collected with CDC light traps or gravid traps, identified and pooled by species. Tissues such as kidney, brain, liver, heart, and spleen can be dissected from dead birds with evidence suggestive of WNV infection and frozen on dry ice and stored at −70° C. prior to RNA isolation.

Procedures for the extraction and purification of WNV RNA from samples are well known in the art. For example, RNA can be isolated from cells using the TRIzol™ reagent (Invitrogen) extraction method. RNA quantity and quality is then determined using, for example, a Nanodrop™ spectrophotometer and an Agilent 2100 bioanalyzer (see also Peirson S N, Butler J N (2007). “RNA extraction from mammalian tissues” Methods Mol. Biol. 362: 315-27, Bird I M (2005) “Extraction of RNA from cells and tissue” Methods Mol. Med. 108: 139-48).

Exemplary methods of extracting WNV RNA from specimens, including mosquitoes and bird tissues, are taught by Shi et al. (1992) entitled “High-Throughput Detection of West Nile Virus RNA” Journal Of Clinical Microbiology 2001, 39(4), p. 1264-1271, Anderson et al. (1999) entitled “Isolation of West Nile virus from mosquitoes, crows, and a Cooper's hawk in Connecticut” Science 286:2331-2333 and Lanciotti et al. (2000) entitled “Rapid detection of West Nile virus from human clinical specimens, field-collected mosquitoes, and avian samples by a TaqMan reverse transcriptase-PCR assay. J. Clin. Microbiol. 38:4066-4071.

In addition, several commercial kits are available for the isolation of WNV RNA. Exemplary kits include, but are not limited to, RNeasy and QIAamp Viral RNA Kit (Qiagen, Valencia, Calif.) and MagMAX™ Viral RNA Isolation Kits (Ambion).

In other embodiments, WNV RNA sequences are obtained by T7 RNA transcription of WNV minigene sequences (SEQ ID NOs: 1-4). An exemplary commercial kit for T7 in vitro transcription is Ambion's MEGAscript® Kit (Catalog No. 1330).

As used herein, a “WNV minigene” refers to selected nucleic acid sequence within a WNV homology region. A minigene sequence can be 25, 50, 75, 100, 150, or 250 nucleotides in length or more.

### Reverse Transcriptase-PCR Amplification of a West Nile Virus RNA Target Nucleic Acid Sequence

One of the most widely used techniques to study WNV gene expression exploits first-strand cDNA for viral RNA sequence(s) to produce a DNA template for amplification by PCR.

The term “reverse transcriptase activity” and “reverse transcription” refers to the enzymatic activity of a class of polymerases characterized as RNA-dependent DNA polymerases that can synthesize a DNA strand (i.e., complementary DNA, cDNA) utilizing an RNA strand as a template.

“Reverse transcriptase-PCR” of “RNA PCR” is a PCR reaction that uses RNA template and a reverse transcriptase, or an enzyme having reverse transcriptase activity, to first generate a single stranded DNA molecule prior to the multiple cycles of DNA-dependent DNA polymerase primer elongation. Multiplex PCR refers to PCR reactions that produce more than one amplified product in a single reaction, typically by the inclusion of more than two primers in a single reaction.

Exemplary reverse transcriptases include, but are not limited to, the Moloney murine leukemia virus (M-MLV) RT as described in U.S. Pat. No. 4,943,531, a mutant form of M-MLV-RT lacking RNase H activity as described in U.S. Pat. No. 5,405,776, bovine leukemia virus (BLV) RT, Rous sarcoma virus (RSV) RT, Avian Myeloblastosis Virus (AMV) RT and reverse transcriptases disclosed in U.S. Pat. No. 7,883,871.

The reverse transcriptase-PCR procedure, carried out as either an end-point or real-time assay, involves two separate molecular syntheses: (i) the synthesis of cDNA from an RNA template; and (ii) the replication of the newly synthesized cDNA through PCR amplification. To attempt to address the technical problems often associated with reverse transcriptase-PCR, a number of protocols have been developed taking into account the three basic steps of the procedure: (a) the denaturation of RNA and the hybridization of reverse primer; (b) the synthesis of cDNA; and (c) PCR amplification.

In the so called “uncoupled” reverse transcriptase-PCR procedure (e.g., two step reverse transcriptase-PCR), reverse transcription is performed as an independent step using the optimal buffer condition for reverse transcriptase activity. Following cDNA synthesis, the reaction is diluted to decrease MgCl₂, and deoxyribonucleoside triphosphate (dNTP) concentrations to conditions optimal for Taq DNA Polymerase activity, and PCR is then carried out according to standard conditions (see U.S. Pat. Nos. 4,683,195 and 4,683,202).

By contrast, “coupled” RT-PCR methods use a common buffer optimized for reverse transcriptase and Taq DNA Polymerase activities. In one version, the annealing of reverse primer is a separate step preceding the addition of enzymes, which are then added to the single reaction vessel. In another version, the reverse transcriptase activity is a component of the thermostable Tth DNA polymerase Annealing and cDNA synthesis are performed in the presence of Mn²⁺ then PCR is carried out in the presence of Mg²⁺ after the removal of Mn²⁺ by a chelating agent.

Finally, the “continuous” method (e.g., one step reverse transcriptase-PCR) integrates the three reverse transcriptase-PCR steps into a single continuous reaction that avoids the opening of the reaction tube for component or enzyme addition. Continuous reverse transcriptase-PCR has been described as a single enzyme system using the reverse transcriptase activity of thermostable Taq DNA Polymerase and Tth polymerase and as a two enzyme system using AMV RT and Taq DNA Polymerase wherein the initial 65° C. RNA denaturation step may be omitted.

In certain embodiments, one or more primers may be labeled.

As used herein, “label,” “detectable label,” or “marker,” or “detectable marker,” which are interchangeably used in the specification, refers to any chemical moiety attached to a nucleotide, nucleotide polymer, or nucleic acid binding factor, wherein the attachment may be covalent or non-covalent. Preferably, the label is detectable and renders the nucleotide or nucleotide polymer detectable to the practitioner of the invention. Detectable labels include luminescent molecules, chemiluminescent molecules, fluorochromes, fluorescent quenching agents, colored molecules, radioisotopes or scintillants. Detectable labels also include any useful linker molecule (such as biotin, avidin, streptavidin, HRP, protein A, protein G, antibodies or fragments thereof, Grb2, polyhistidine, Ni²⁺, FLAG tags, myc tags), heavy metals, enzymes (examples include alkaline phosphatase, peroxidase and luciferase), electron donors/acceptors, acridinium esters, dyes and calorimetric substrates. It is also envisioned that a change in mass may be considered a detectable label, as is the case of surface plasmon resonance detection. The skilled artisan would readily recognize useful detectable labels that are not mentioned above, which may be employed in the operation of the present invention.

One step reverse transcriptase-PCR provides several advantages over uncoupled reverse transcriptase-PCR. One step reverse transcriptase-PCR requires less handling of the reaction mixture reagents and nucleic acid products than uncoupled reverse transcriptase-PCR (e.g., opening of the reaction tube for component or enzyme addition in between the two reaction steps), and is therefore less labor intensive, reducing the required number of person hours. One step reverse transcriptase-PCR also requires less sample, and reduces the risk of contamination. The sensitivity and specificity of one-step reverse transcriptase-PCR has proven well suited for studying expression levels of one to several genes in a given sample or the detection of pathogen RNA. Typically, this procedure has been limited to use of gene-specific primers to initiate cDNA synthesis.

The ability to measure the kinetics of a PCR reaction by on-line detection in combination with these reverse transcriptase-PCR techniques allows for the accurate and precise quantitation of RNA copy number with high sensitivity. This has become possible by detecting the reverse transcriptase-PCR product through fluorescence monitoring and measurement of PCR product during the amplification process by fluorescent dual-labeled hybridization probe technologies, such as the 5′ fluorogenic nuclease assay (“TaqMan'”) or endonuclease assay (“CataCleave™”), discussed below.

PCR Amplification of West Nile Virus cDNA Nucleic Acid Sequences

Once the primers are selected and the cDNA template in a test sample is prepared (see above), nucleic acid amplification can be accomplished by a variety of methods, including the polymerase chain reaction (PCR), nucleic acid sequence based amplification (NASBA), ligase chain reaction (LCR), and rolling circle amplification (RCA). The polymerase chain reaction (PCR) is the method most commonly used to amplify specific target DNA sequences.

“Polymerase chain reaction,” or “PCR,” generally refers to a method for amplification of a desired nucleotide sequence in vitro. The procedure is described in detail in U.S. Pat. Nos. 4,683,202, 4,683,195, 4,800,159, and 4,965,188, the contents of which are hereby incorporated herein in their entirety. Generally, the PCR process consists of introducing a molar excess of two or more extendable oligonucleotide primers to a reaction mixture comprising the desired target sequence(s), where the primers are complementary to opposite strands of the double stranded target sequence. The reaction mixture is subjected to a program of thermal cycling in the presence of a DNA polymerase, resulting in the amplification of the desired target sequence flanked by the DNA primers.

Primers which can be used in the embodiments may have a DNA sequence of SEQ ID NOs. 5-8.

A probe which can be used in the embodiments of the instant application (sometimes referred to as “CataCleave™ probe”) may have the following sequence:

(SEQ ID NO: 9)
FAM/ATG ATT GAC CCrU rUrUrU CAG TTG GGC CTT/IABlk
FQ

As used herein, the term “PCR fragment” or “amplicon” refers to a polynucleotide molecule (or collectively the plurality of molecules) produced following the amplification of a particular target nucleic acid. A PCR fragment is typically, but not exclusively, a DNA PCR fragment. A PCR fragment can be single-stranded or double-stranded, or in a mixture thereof in any concentration ratio. A PCR fragment can be 100-500 nucleotides or more in length.

An amplification “buffer” is a compound added to an amplification reaction which modifies the stability, activity, and/or longevity of one or more components of the amplification reaction by regulating the amplification reaction. The buffering agents of the invention are compatible with PCR amplification and RNase H cleavage activity. Examples of buffers include, but are not limited to, HEPES ((4-(2-hydroxyethyl)-1-piperazineethanesulfonic acid), MOPS (3-(N-morpholino)-propanesulfonic acid), and acetate or phosphate containing buffers and the like. In addition, PCR buffers may generally contain up to about 70 mM KCl and about 1.5 mM or higher MgCl₂, to about 50-200 μM each of dATP, dCTP, dGTP and dTTP. The buffers of the invention may contain additives to optimize efficient reverse transcriptase-PCR or PCR reactions.

An additive is a compound added to a composition which modifies the stability, activity, and/or longevity of one or more components of the composition. In certain embodiments, the composition is an amplification reaction composition. In certain embodiments, an additive inactivates contaminant enzymes, stabilizes protein folding, and/or decreases aggregation. Exemplary additives that may be included in an amplification reaction include, but are not limited to, betaine, formamide, KCl, CaCl₂, MgOAc, MgCl₂, NaCl, NH₄OAc, NaI, Na(CO₃)₂, LiCl, MnOAc, NMP, trehalose, demethylsulfoxide (“DMSO”), glycerol, ethylene glycol, dithiothreitol (“DTT”), pyrophosphatase (including, but not limited to Thermoplasma acidophilum inorganic pyrophosphatase (“TAP”)), bovine serum albumin (“BSA”), propylene glycol, glycinamide, CHES, Percoll, aurintricarboxylic acid, Tween 20, Tween 21, Tween 40, Tween 60, Tween 85, Brij 30, NP-40, Triton X-100, CHAPS, CHAPSO, Mackernium, LDAO (N-dodecyl-N,N-dimethylamine-N-oxide), Zwittergent 3-10, Xwittergent 3-14, Xwittergent SB 3-16, Empigen, NDSB-20, T4G32, E. Coli SSB, RecA, nicking endonucleases, 7-deazaG, dUTP, anionic detergents, cationic detergents, non-ionic detergents, zwittergent, sterol, osmolytes, cations, and any other chemical, protein, or cofactor that may alter the efficiency of amplification. In certain embodiments, two or more additives are included in an amplification reaction. Additives may be optionally added to improve selectivity of primer annealing provided the additives do not interfere with the activity of RNase H.

As used herein, the term “thermostable,” as applied to an enzyme, refers to an enzyme that retains its biological activity at elevated temperatures (e.g., at 55° C. or higher), or retains its biological activity following repeated cycles of heating and cooling. Thermostable polynucleotide polymerases find particular use in PCR amplification reactions.

As used herein, a “thermostable polymerase” is an enzyme that is relatively stable to heat and eliminates the need to add enzyme prior to each PCR cycle. Non-limiting examples of thermostable polymerases may include polymerases isolated from the thermophilic bacteria Thermus aquaticus (Taq polymerase), Thermus thermophilus (Tth polymerase), Thermococcus litoralis (Tli or VENT polymerase), Pyrococcus furiosus (Pfu or DEEPVENT polymerase), Pyrococcus woosii (Pwo polymerase) and other Pyrococcus species, Bacillus stearothermophilus (Bst polymerase), Sulfolobus acidocaldarius (Sac polymerase), Thermoplasma acidophilum (Tac polymerase), Thermus rubber (Tru polymerase), Thermus brockianus (DYNAZYME polymerase) Thermotoga neapolitana (Tne polymerase), Thermotoga maritime (Tma) and other species of the Thermotoga genus (Tsp polymerase), and Methanobacterium thermoautotrophicum (Mth polymerase). The PCR reaction may contain more than one thermostable polymerase enzyme with complementary properties leading to more efficient amplification of target sequences. For example, a nucleotide polymerase with high processivity (the ability to copy large nucleotide segments) may be complemented with another nucleotide polymerase with proofreading capabilities (the ability to correct mistakes during elongation of target nucleic acid sequence), thus creating a PCR reaction that can copy a long target sequence with high fidelity. The thermostable polymerase may be used in its wild type form. Alternatively, the polymerase may be modified to contain a fragment of the enzyme or to contain a mutation that provides beneficial properties to facilitate the PCR reaction. In one embodiment, the thermostable polymerase may be Taq polymerase. Many variants of Taq polymerase with enhanced properties are known and include AmpliTaq, AmpliTaq Stoffel fragment, SuperTaq, SuperTaq plus, LA Taq, LApro Taq, and EX Taq.

### Real-Time PCR Using a CataCleave™ Probe

Post amplification amplicon detection can be both laborious and time consuming. Real-time methods have been developed to monitor amplification during the PCR process. These methods typically employ fluorescently labeled probes that bind to the newly synthesized DNA or dyes whose fluorescence emission is increased when intercalated into double stranded DNA.

The probes are generally designed so that donor emission is quenched in the absence of target by fluorescence resonance energy transfer (FRET) between two chromophores. The donor chromophore, in its excited state, may transfer energy to an acceptor chromophore when the pair is in close proximity. This transfer is always non-radiative and occurs through dipole-dipole coupling. Any process that sufficiently increases the distance between the chromophores will decrease FRET efficiency such that the donor chromophore emission can be detected radiatively. Common donor chromophores include FAM, TAMRA, VIC, JOE, Cy3, Cy5, and Texas Red.) Acceptor chromophores are chosen so that their excitation spectra overlap with the emission spectrum of the donor. An example of such a pair is FAM-TAMRA. There are also non fluorescent acceptors that will quench a wide range of donors. Other examples of appropriate donor-acceptor FRET pairs will be known to those skilled in the art.

Common examples of FRET probes that can be used for real-time detection of PCR include molecular beacons (e.g., U.S. Pat. No. 5,925,517), TaqMan™ probes (e.g., U.S. Pat. Nos. 5,210,015 and 5,487,972), and CataCleave™ probes (e.g., U.S. Pat. No. 5,763,181). The molecular beacon is a single stranded oligonucleotide designed so that in the unbound state the probe forms a secondary structure where the donor and acceptor chromophores are in close proximity and donor emission is reduced. At the proper reaction temperature the beacon unfolds and specifically binds to the amplicon. Once unfolded the distance between the donor and acceptor chromophores increases such that FRET is reversed and donor emission can be monitored using specialized instrumentation. TaqMan™ and CataCleave™ technologies differ from the molecular beacon in that the FRET probes employed are cleaved such that the donor and acceptor chromophores become sufficiently separated to reverse FRET.

TaqMan™ technology employs a single-stranded oligonucleotide probe that is labeled at the 5′ end with a donor chromophore and at the 3′ end with an acceptor chromophore. The DNA polymerase used for amplification must contain a 5′->3′ exonuclease activity. The TaqMan™ probe binds to one strand of the amplicon at the same time that the primer binds. As the DNA polymerase extends the primer the polymerase will eventually encounter the bound TaqMan™ probe. At this time the exonuclease activity of the polymerase will sequentially degrade the TaqMan™ probe starting at the 5′ end. As the probe is digested the mononucleotides comprising the probe are released into the reaction buffer. The donor diffuses away from the acceptor and FRET is reversed. Emission from the donor is monitored to identify probe cleavage. Because of the way TaqMan™ works a specific amplicon can be detected only once for every cycle of PCR. Extension of the primer through the TaqMan™ target site generates a double stranded product that prevents further binding of TaqMan™ probes until the amplicon is denatured in the next PCR cycle.

U.S. Pat. No. 5,763,181, the contents of which are incorporated herein by reference, describes another real-time detection method (referred to as “CataCleave™”). CataCleave™ technology differs from TaqMan™ in that cleavage of the probe is accomplished by a second enzyme that does not have polymerase activity. The CataCleave™ probe has a sequence within the molecule which is a target of an endonuclease, such as, for example a restriction enzyme or RNAase. In one example, the CataCleave™ probe has a chimeric structure where the 5′ and 3′ ends of the probe are constructed of DNA and the cleavage site contains RNA. The DNA sequence portions of the probe are labeled with a FRET pair either at the ends or internally. The PCR reaction includes an RNase H enzyme that will specifically cleave the RNA sequence portion of a RNA-DNA duplex. After cleavage, the two halves of the probe dissociate from the target amplicon at the reaction temperature and diffuse into the reaction buffer. As the donor and acceptors separate FRET is reversed in the same way as the TaqMan™ probe and donor emission can be monitored. Cleavage and dissociation regenerates a site for further CataCleave™ binding. In this way it is possible for a single amplicon to serve as a target or multiple rounds of probe cleavage until the primer is extended through the CataCleave™ probe binding site.

### Labeling of a CataCleave™ Probe

The term “probe” comprises a polynucleotide that comprises a specific portion designed to hybridize in a sequence-specific manner with a complementary region of a specific nucleic acid sequence, e.g., a target nucleic acid sequence. In one embodiment, the oligonucleotide probe is in the range of 15-60 nucleotides in length. More preferably, the oligonucleotide probe is in the range of 18-30 nucleotides in length. The precise sequence and length of an oligonucleotide probe of the invention depends in part on the nature of the target polynucleotide to which it binds. The binding location and length may be varied to achieve appropriate annealing and melting properties for a particular embodiment. Guidance for making such design choices can be found in many of the references describing TaqMan™ assays or CataCleave™, described in U.S. Pat. Nos. 5,763,181, 6,787,304, and 7,112,422, the contents of which are incorporated herein by reference.

In certain embodiments, the probe is “substantially complementary” to the target nucleic acid sequence.

As used herein, the term “substantially complementary” refers to two nucleic acid strands that are sufficiently complimentary in sequence to anneal and form a stable duplex. The complementarity does not need to be perfect; there may be any number of base pair mismatches, for example, between the two nucleic acids. However, if the number of mismatches is so great that no hybridization can occur under even the least stringent hybridization conditions, the sequence is not a substantially complementary sequence. When two sequences are referred to as “substantially complementary” herein, it means that the sequences are sufficiently complementary to each other to hybridize under the selected reaction conditions. The relationship of nucleic acid complementarity and stringency of hybridization sufficient to achieve specificity is well known in the art. Two substantially complementary strands can be, for example, perfectly complementary or can contain from 1 to many mismatches so long as the hybridization conditions are sufficient to allow, for example discrimination between a pairing sequence and a non-pairing sequence. Accordingly, “substantially complementary” sequences can refer to sequences with base-pair complementarity of 100, 95, 90, 80, 75, 70, 60, 50 percent or less, or any number in between, in a double-stranded region.

As used herein, a “selected region” refers to a polynucleotide sequence of a target DNA or cDNA that anneals with the RNA sequences of a probe. In one embodiment, a “selected region” of a target DNA or cDNA can be from 1, 2, 3, 4, 5, 6, 7, 8, 9, 10, 11, 12, 13, 14, 15, 16, 17, 18, 19, 20, 21, 22, 23, 24, 25 or more nucleotides in length.

As used herein, RNase H cleavage refers to the cleavage of the RNA moiety of the Catacleave™ probe that is entirely complimentary to and hybridizes with a target DNA sequence to form an RNA:DNA heteroduplex.

As used herein, “label” or “detectable label” of the CataCleave™ probe refers to any label comprising a fluorochrome compound that is attached to the probe by covalent or non-covalent means.

As used herein, “fluorochrome” refers to a fluorescent compound that emits light upon excitation by light of a shorter wavelength than the light that is emitted. The term “fluorescent donor” or “fluorescence donor” refers to a fluorochrome that emits light that is measured in the assays described in the present invention. More specifically, a fluorescent donor provides energy that is absorbed by a fluorescence acceptor. The term “fluorescent acceptor” or “fluorescence acceptor” refers to either a second fluorochrome or a quenching molecule that absorbs energy emitted from the fluorescence donor. The second fluorochrome absorbs the energy that is emitted from the fluorescence donor and emits light of longer wavelength than the light emitted by the fluorescence donor. The quenching molecule absorbs energy emitted by the fluorescence donor.

Any luminescent molecule, preferably a fluorochrome and/or fluorescent quencher may be used in the practice of this invention, including, for example, Alexa Fluor™ 350, Alexa Fluor™ 430, Alexa Fluor™ 488, Alexa Fluor™ 532, Alexa Fluor™ 546, Alexa Fluor™ 568, Alexa Fluor™ 594, Alexa Fluor™ 633, Alexa Fluor™ 647, Alexa Fluor™ 660, Alexa Fluor™ 680, 7-diethylaminocoumarin-3-carboxylic acid, Fluorescein, Oregon Green 488, Oregon Green 514, Tetramethylrhodamine, Rhodamine X, Texas Red dye, QSY 7, QSY33, Dabcyl, BODIPY FL, BODIPY 630/650, BODIPY 6501665, BODIPY TMR-X, BODIPY TR-X, Dialkylaminocoumarin, Cy5.5, Cy5, Cy3.5, Cy3, DTPA(Eu³⁺)-AMCA and TTHA(Eu³⁺)AMCA.

In one embodiment, the 3′ terminal nucleotide of the oligonucleotide probe is blocked or rendered incapable of extension by a nucleic acid polymerase. Such blocking is conveniently carried out by the attachment of a reporter or quencher molecule to the terminal 3′ position of the probe.

In one embodiment, reporter molecules are fluorescent organic dyes derivatized for attachment to the terminal 3′ or terminal 5′ ends of the probe via a linking moiety. Preferably, quencher molecules are also organic dyes, which may or may not be fluorescent, depending on the embodiment of the invention. For example, in a preferred embodiment of the invention, the quencher molecule is fluorescent. Generally whether the quencher molecule is fluorescent or simply releases the transferred energy from the reporter by non-radiative decay, the absorption band of the quencher should substantially overlap the fluorescent emission band of the reporter molecule. Non-fluorescent quencher molecules that absorb energy from excited reporter molecules, but which do not release the energy radiatively, are referred to in the application as chromogenic molecules.

Exemplary reporter-quencher pairs may be selected from xanthene dyes, including fluoresceins, and rhodamine dyes. Many suitable forms of these compounds are widely available commercially with substituents on their phenyl moieties which can be used as the site for bonding or as the bonding functionality for attachment to an oligonucleotide. Another group of fluorescent compounds are the naphthylamines, having an amino group in the alpha or β position. Included among such naphthylamino compounds are 1-dimethylaminonaphthyl-5-sulfonate, 1-anilino-8-naphthalene sulfonate and 2-p-touidinyl6-naphthalene sulfonate. Other dyes include 3-phenyl-7-isocyanatocoumarin, acridines, such as 9-isothiocyanatoacridine and acridine orange, N-(p-(2-benzoxazolyl)phenyl)maleimide, benzoxadiazoles, stilbenes, pyrenes, and the like.

In one embodiment, reporter and quencher molecules are selected from fluorescein and rhodamine dyes.

There are many linking moieties and methodologies for attaching reporter or quencher molecules to the 5′ or 3′ termini of oligonucleotides, as exemplified by the following references: Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19: 3019 (1991) (3′ sulfhydryl); Giusti et al., PCR Methods and Applications, 2: 223-227 (1993) and Fung et al., U.S. Pat. No. 4,757,141 (5′ phosphoamino group via Aminolink™ II available from Applied Biosystems, Foster City, Calif.) Stabinsky, U.S. Pat. No. 4,739,044 (3′ aminoalkylphosphoryl group); Agrawal et al., Tetrahedron Letters, 31: 1543-1546 (1990) (attachment via phosphoramidate linkages); Sproat et al., Nucleic Acids Research, 15: 4837 (1987) (5′ mercapto group); Nelson et al., Nucleic Acids Research, 17: 7187-7194 (1989) (3′ amino group); and the like.

Rhodamine and fluorescein dyes are also conveniently attached to the 5′ hydroxyl of an oligonucleotide at the conclusion of solid phase synthesis by way of dyes derivatized with a phosphoramidite moiety, e.g., Woo et al., U.S. Pat. No. 5,231,191; and Hobbs, Jr., U.S. Pat. No. 4,997,928.

### Attachment of a CataCleave™ Probe to a Solid Support

In one embodiment, the oligonucleotide probe can be attached to a solid support. Different probes may be attached to the solid support and may be used to simultaneously detect different target sequences in a sample. Reporter molecules having different fluorescence wavelengths can be used on the different probes, thus enabling hybridization to the different probes to be separately detected.

Examples of preferred types of solid supports for immobilization of the oligonucleotide probe include controlled pore glass, glass plates, polystyrene, avidin coated polystyrene beads cellulose, nylon, acrylamide gel and activated dextran, controlled pore glass (CPG), glass plates and high cross-linked polystyrene. These solid supports are preferred for hybridization and diagnostic studies because of their chemical stability, ease of functionalization and well defined surface area. Solid supports such as controlled pore glass (500 \acute{Å}, 1000 \acute{Å}) and non-swelling high cross-linked polystyrene (1000 \acute{Å}) are particularly preferred in view of their compatibility with oligonucleotide synthesis.

The oligonucleotide probe may be attached to the solid support in a variety of manners. For example, the probe may be attached to the solid support by attachment of the 3′ or 5′ terminal nucleotide of the probe to the solid support. However, the probe may be attached to the solid support by a linker which serves to distance the probe from the solid support. The linker is most preferably at least 30 atoms in length, more preferably at least 50 atoms in length.

Hybridization of a probe immobilized to a solid support generally requires that the probe be separated from the solid support by at least 30 atoms, more-preferably at least 50 atoms. In order to achieve this separation, the linker generally includes a spacer positioned between the linker and the 3′ nucleoside. For oligonucleotide synthesis, the linker arm is usually attached to the 3′-OH of the 3′ nucleoside by an ester linkage which can be cleaved with basic reagents to free the oligonucleotide from the solid support.

A wide variety of linkers are known in the art which may be used to attach the oligonucleotide probe to the solid support. The linker may be formed of any compound which does not significantly interfere with the hybridization of the target sequence to the probe attached to the solid support. The linker may be formed of a homopolymeric oligonucleotide which can be readily added on to the linker by automated synthesis. Alternatively, polymers such as functionalized polyethylene glycol can be used as the linker. Such polymers are preferred over homopolymeric oligonucleotides because they do not significantly interfere with the hybridization of probe to the target oligonucleotide. Polyethylene glycol is particularly preferred because it is commercially available, soluble in both organic and aqueous media, easy to functionalize, and completely stable under oligonucleotide synthesis and post-synthesis conditions.

The linkages between the solid support, the linker and the probe are preferably not cleaved during removal of base protecting groups under basic conditions at high temperature. Examples of preferred linkages include carbamate and amide linkages. Immobilization of a probe is well known in the art and one skilled in the art may determine the immobilization conditions.

According to one embodiment of the method, the CataCleave™ probe is immobilized on a solid support. The CataCleave™ probe comprises a detectable label and DNA and RNA nucleic acid sequences, wherein the probe's RNA nucleic acid sequences are entirely complementary to a selected region of the target DNA sequence and the probe's DNA nucleic acid sequences are substantially complementary to DNA sequences adjacent to the selected region of the target DNA sequence. The probe is then contacted with a sample of nucleic acids in the presence of RNase H and under conditions where the RNA sequences within the probe can form a RNA:DNA heteroduplex with the complementary DNA sequences. RNase H cleavage of the RNA sequences within the RNA:DNA heteroduplex results in a real-time increase in the emission of a signal from the label on the probe indicating the presence of WNV nucleic acid sequences in the sample.

RNase H cleavage of the Catacleave™ Probe

RNase H hydrolyzes RNA in RNA-DNA hybrids. First identified in calf thymus, RNase H has subsequently been described in a variety of organisms. Indeed, RNase H activity appears to be ubiquitous in eukaryotes and bacteria. Although RNase Hs form a family of proteins of varying molecular weight and nucleolytic activity, substrate requirements appear to be similar for the various isotypes. For example, most RNase Hs studied to date function as endonucleases and require divalent cations (e.g., Mg²⁺, Mn²⁺) to produce cleavage products with 5′ phosphate and 3′ hydroxyl termini.

In prokaryotes, RNase H have been cloned and extensively characterized (see Crooke, et al., (1995) Biochem J, 312 (Pt 2), 599-608; Lima, et al., (1997) J Biol Chem, 272, 27513-27516; Lima, et al., (1997) Biochemistry, 36, 390-398; Lima, et al., (1997) J Biol Chem, 272, 18191-18199; Lima, et al., (2007) Mol Pharmacol, 71, 83-91; Lima, et al., (2007) Mol Pharmacol, 71, 73-82; Lima, et al., (2003) J Biol Chem, 278, 14906-14912; Lima, et al., (2003) J Biol Chem, 278, 49860-49867; Itaya, M., Proc. Natl. Acad. Sci. USA, 1990, 87, 8587-8591). For example, E. coli RNase HII is 213 amino acids in length whereas RNase HI is 155 amino acids long. E. coli RNase HII displays only 17% homology with E. coli RNase HI. An RNase H cloned from S. typhimurium differed from E. coli RNase HI in only 11 positions and was 155 amino acids in length (Itaya, M. and Kondo K., Nucleic Acids Res., 1991, 19, 4443-4449).

Proteins that display RNase H activity have also been cloned and purified from a number of viruses, other bacteria and yeast (Wintersberger, U. Pharmac. Ther., 1990, 48, 259-280). In many cases, proteins with RNase H activity appear to be fusion proteins in which RNase H is fused to the amino or carboxy end of another enzyme, often a DNA or RNA polymerase. The RNase H domain has been consistently found to be highly homologous to E. coli RNase HI, but because the other domains vary substantially, the molecular weights and other characteristics of the fusion proteins vary widely.

In higher eukaryotes two classes of RNase H have been defined based on differences in molecular weight, effects of divalent cations, sensitivity to sulfhydryl agents and immunological cross-reactivity (Busen et al., Eur. J. Biochem., 1977, 74, 203-208). RNase HI enzymes are reported to have molecular weights in the 68-90 kDa range, be activated by either Mn²⁺ or Mg²⁺ and be insensitive to sulfhydryl agents. In contrast, RNase HII enzymes have been reported to have molecular weights ranging from 31-45 kDa, to require Mg²⁺ to be highly sensitive to sulfhydryl agents and to be inhibited by Mn²⁺ (Busen, W., and Hausen, P., Eur. J. Biochem., 1975, 52, 179-190; Kane, C. M., Biochemistry, 1988, 27, 3187-3196; Busen, W., J. Biol. Chem., 1982, 257, 7106-7108)

An enzyme with RNase HII characteristics has also been purified to near homogeneity from human placenta (Frank et al., Nucleic Acids Res., 1994, 22, 5247-5254). This protein has a molecular weight of approximately 33 kDa and is active in a pH range of 6.5-10, with a pH optimum of 8.5-9. The enzyme requires Mg²⁺ and is inhibited by Mn²⁺ and n-ethyl maleimide. The products of cleavage reactions have 3′ hydroxyl and 5′ phosphate termini.

A detailed comparison of RNases from different species is reported in Ohtani N, Haruki M, Morikawa M, Kanaya S. J Biosci Bioeng. 1999; 88(1):12-9.

Examples of RNase H enzymes, which may be employed in the embodiments, also include, but are not limited to, thermostable RNase H enzymes isolated from thermophilic organisms such as Pyrococcus furiosus, Pyrococcus horikoshi, Thermococcus litoralis or Thermus thermophilus.

Other RNase H enzymes that may be employed in the embodiments are described in, for example, U.S. Pat. No. 7,422,888 to Uemori or the published U.S. Patent Application No. 2009/0325169 to Walder, the contents of which are incorporated herein by reference.

In one embodiment, an RNase H enzyme is a thermostable RNase H with 40%, 50%, 60%, 70%, 80%, 90%, 95% or 99% homology with the amino acid sequence of Pfu RNase HII (SEQ ID NO: 10), shown below.

| (SEQ ID NO: 10) | |
|---|---|
| MKIGGIDEAG RGPAIGPLVV ATVVVDEKNI EKLRNIGVKD SKQLTPHERK NLFSQITSIA | 60 |
| DDYKIVIVSP EEIDNRSGTM NELEVEKFAL ALNSLQIKPA LIYADAADVD ANRFASLIER | 120 |
| RLNYKAKIIA EHKADAKYPV VSAASILAKV VRDEEIEKLK KQYGDFGSGY PSDPKTKKWL | 180 |
| EEYYKKHNSF PPIVRRTWET VRKIEESIKA KKSQLTLDKF FKKP | |

The homology can be determined using, for example, a computer program DNASIS-Mac (Takara Shuzo), a computer algorithm FASTA (version 3.0; Pearson, W. R. et al., Pro. Natl. Acad. Sci., 85:2444-2448, 1988) or a computer algorithm BLAST (version 2.0, Altschul et al., Nucleic Acids Res. 25:3389-3402, 1997)

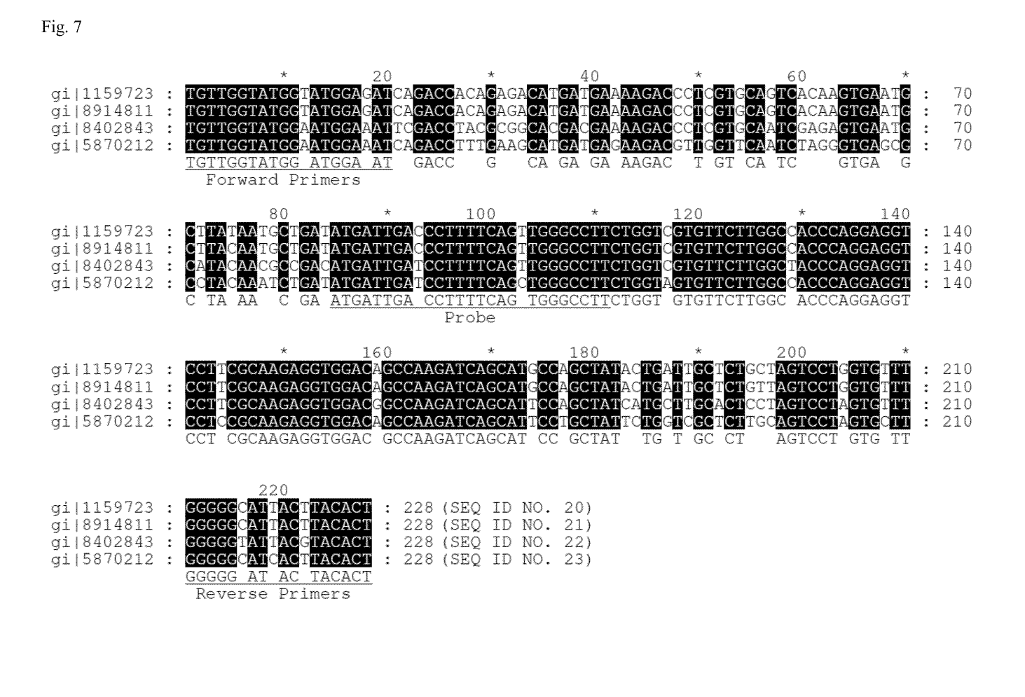

In another embodiment, an RNase H enzyme is a thermostable RNase H with at least one or more homology regions 1-4 corresponding to positions 5-20, 33-44, 132-150, and 158-173 of SEQ ID NO: 10. These homology regions were defined by sequence alignment of Pyrococcus furiosis, Pyrococcus horikoshi, Thermococcus kodakarensis, Archeoglobus profundus, Archeoglobus fulgidis, Thermococcus celer and Thermococcus litoralis RNase HII polypeptide sequences (see FIG. 7).

HOMOLOGY REGION 1:
GIDEAG RGPAIGPLVV (SEQ ID NO: 11; corresponding
to positions 5-20 of SEQ ID NO: 10)
HOMOLOGY REGION 2:
LRNIGVKD SKQL (SEQ ID NO: 12; corresponding to
positions 33-44 of SEQ ID NO: 10)
HOMOLOGY REGION 3:
HKADAKYPV VSAASILAKV (SEQ ID NO: 13; correspond-
ing to positions 132-150 of SEQ ID NO: 10)
HOMOLOGY REGION 4:
KLK KQYGDFGSGY PSD (SEQ ID NO: 14; corresponding
to positions 158-173 of SEQ ID NO: 10)

In one embodiment, an RNase H enzyme is a thermostable RNase H with at least one of the homology regions having 50%, 60%. 70%, 80%, 90% sequence identity with a polypeptide sequence of SEQ ID NOs: 11, 12, 13 or 14.

In another embodiment, an RNase H enzyme is a thermostable RNase H with 40%, 50%, 60%, 70%, 80%, 90%, 95% or 99% homology with the amino acid sequence of Thermus thermophilus RNase HI (SEQ ID NO: 15), shown below.

(SEQ ID NO: 15)
MNPSPRKRVA LFTDGACLGN PGPGGWAALL RFHAHEKLLS GGEACTTNNR MELKAAIEGL
KALKEPCEVD LYTDSHYLKK AFTEGWLEGW RKRGWRTAEG KPVKNRDLWE ALLLAMAPHR
VRFHFVKGHT GHPENERVDR EARRQAQSQA KTPCPPRAPT LFHEEA

In another embodiment, an RNase H enzyme is a thermostable RNase H with at least one or more homology regions 5-8 corresponding to positions 23-48, 62-69, 117-121 and 141-152 of SEQ ID NO: 15. These homology regions were defined by sequence alignment of Haemophilus influenzae, Thermus thermophilis, Thermus acquaticus, Salmonella enterica and Agrobacterium tumefaciens RNase HI polypeptide sequences (see FIG. 11).

HOMOLOGY REGION 5:
K*V*LFTDG*C*GNPG*GG*ALLRY (SEQ ID NO: 16;
corresponding to positions 23-48 of SEQ ID NO: 15)
HOMOLOGY REGION 6:
TTNNRMEL (SEQ ID NO: 17; corresponding to
positions 62-69 of SEQ ID NO: 15)
HOMOLOGY REGION 7:
KPVKN (SEQ ID NO: 18; corresponding to positions
117-121 of SEQ ID NO: 15)
HOMOLOGY REGION 8:
FVKGH*GH*ENE (SEQ ID NO: 19; corresponding to
positions 141-152 of SEQ ID NO: 15)

In another embodiment, an RNase H enzyme is a thermostable RNase H with at least one of the homology regions 4-8 having 50%, 60%. 70%, 80%, 90% sequence identity with a polypeptide sequence of SEQ ID NOs: 16, 17, 18 or 19.

The terms “sequence identity,” as used herein, refers to the extent that sequences are identical or functionally or structurally similar on a amino acid to amino acid basis over a window of comparison. Thus, a “percentage of sequence identity”, for example, can be calculated by comparing two optimally aligned sequences over the window of comparison, determining the number of positions at which the identical amino acid occurs in both sequences to yield the number of matched positions, dividing the number of matched positions by the total number of positions in the window of comparison (i.e., the window size), and multiplying the result by 100 to yield the percentage of sequence identity.

In certain embodiments, the RNase H can be modified to produce a hot start “inducible” RNase H.

The term “modified RNase H,” as used herein, can be an RNase H reversely coupled to or reversely bound to an inhibiting factor that causes the loss of the endonuclease activity of the RNase H. Release or decoupling of the inhibiting factor from the RNase H restores at least partial or full activity of the endonuclease activity of the RNase H. About 30-100% of its activity of an intact RNase H may be sufficient. The inhibiting factor may be a ligand or a chemical modification. The ligand can be an antibody, an aptamer, a receptor, a cofactor, or a chelating agent. The ligand can bind to the active site of the RNase H enzyme thereby inhibiting enzymatic activity or it can bind to a site remote from the RNase's active site. In some embodiments, the ligand may induce a conformational change. The chemical modification can be a cross-linking (for example, by formaldehyde) or acylation. The release or decoupling of the inhibiting factor from the RNase H may be accomplished by heating a sample or a mixture containing the coupled RNase H (inactive) to a temperature of about 65° C. to about 95° C. or higher, and/or lowering the pH of the mixture or sample to about 7.0 or lower.

As used herein, a hot start “inducible” RNase H activity refers to the herein described modified RNase H that has an endonuclease catalytic activity that can be regulated by association with a ligand. Under permissive conditions, the RNase H endonuclease catalytic activity is activated whereas at non-permissive conditions, this catalytic activity is inhibited. In some embodiments, the catalytic activity of a modified RNase H can be inhibited at temperature conducive for reverse transcription, i.e. about 42° C., and activated at more elevated temperatures found in PCR reactions, i.e. about 65° C. to 95° C. A modified RNase H with these characteristics is said to be “heat inducible.”

In other embodiments, the catalytic activity of a modified RNase H can be regulated by changing the pH of a solution containing the enzyme.

As used herein, a “hot start” enzyme composition refers to compositions having an enzymatic activity that is inhibited at non-permissive temperatures, i.e. from about 25° C. to about 45° C. and activated at temperatures compatible with a PCR reaction, e.g. about 55° C. to about 95° C. In certain embodiment, a “hot start” enzyme composition may have a ‘hot start’ RNase H and/or a ‘hot start’ thermostable DNA polymerase that are known in the art.

Cross-linking of RNase H enzymes can be performed using, for example, formaldehyde. In one embodiment, a thermostable RNase H is subjected to controlled and limited crosslinking using formaldehyde. By heating an amplification reaction composition, which comprises the modified RNase H in an active state, to a temperature of about 95° C. or higher for an extended time, for example about 15 minutes, the cross-linking is reversed and the RNase H activity is restored.

In general, the lower the degree of cross-linking, the higher the endonuclease activity of the enzyme is after reversal of cross-linking. The degree of cross-linking may be controlled by varying the concentration of formaldehyde and the duration of cross-linking reaction. For example, about 0.2% (w/v), about 0.4% (w/v), about 0.6% (w/v), or about 0.8% (w/v) of formaldehyde may be used to crosslink an RNase H enzyme. About 10 minutes of cross-linking reaction using 0.6% formaldehyde may be sufficient to inactivate RNase HII from Pyrococcus furiosus.

The cross-linked RNase H does not show any measurable endonuclease activity at about 37° C. In some cases, a measurable partial reactivation of the cross-linked RNase H may occur at a temperature of around 50° C., which is lower than the PCR denaturation temperature. To avoid such unintended reactivation of the enzyme, it may be required to store or keep the modified RNase H at a temperature lower than 50° C. until its reactivation.

In general, PCR requires heating the amplification composition at each cycle to about 95° C. to denature the double stranded target sequence which will also release the inactivating factor from the RNase H, partially or fully restoring the activity of the enzyme.

RNase H may also be modified by subjecting the enzyme to acylation of lysine residues using an acylating agent, for example, a dicarboxylic acid. Acylation of RNase H may be performed by adding cis-aconitic anhydride to a solution of RNase H in an acylation buffer and incubating the resulting mixture at about 1-20° C. for 5-30 hours. In one embodiment, the acylation may be conducted at around 3-8° C. for 18-24 hours. The type of the acylation buffer is not particularly limited. In an embodiment, the acylation buffer has a pH of between about 7.5 to about 9.0.

The activity of acylated RNase H can be restored by lowering the pH of the amplification composition to about 7.0 or less. For example, when Tris buffer is used as a buffering agent, the composition may be heated to about 95° C., resulting in the lowering of pH from about 8.7 (at 25° C.) to about 6.5 (at 95° C.).

The duration of the heating step in the amplification reaction composition may vary depending on the modified RNase H, the buffer used in the PCR, and the like. However, in general, heating the amplification composition to 95° C. for about 30 seconds-4 minutes is sufficient to restore RNase H activity. In one embodiment, using a commercially available buffer and one or more non-ionic detergents, full activity of Pyrococcus furiosus RNase HII is restored after about 2 minutes of heating.

RNase H activity may be determined using methods that are well in the art. For example, according to a first method, the unit activity is defined in terms of the acid-solubilization of a certain number of moles of radiolabeled polyadenylic acid in the presence of equimolar polythymidylic acid under defined assay conditions (see Epicentre Hybridase thermostable RNase HI). In the second method, unit activity is defined in terms of a specific increase in the relative fluorescence intensity of a reaction containing equimolar amounts of the probe and a complementary template DNA under defined assay conditions.

### Real-Time Detection of West Nile Virus Target Nucleic Acid Sequences Using a CataCleave™ Probe

The labeled oligonucleotide probe may be used as a probe for the real-time detection of WNV target nucleic acid sequences in a sample.

A CataCleave oligonucleotide probe is first synthesized with DNA and RNA sequences that are complimentary to WNV nucleic acid sequences found within a PCR amplicon comprising a selected WNV target sequence. In one embodiment, the probe is labeled with a FRET pair, for example, a fluorescein molecule at one end of the probe and a non-fluorescent quencher molecule at the other end.

In certain embodiments, cells, such as blood cells, suspected of being infected with WNV are lysed, total RNA is extracted from the cells, reverse transcribed and subjected to real-time Catacleave™-PCR for the detection of WNV RNA sequences according to the methods described herein. If WNV RNA sequences are present in the sample, during the reverse transcription real-time PCR reaction, the labeled probe can hybridize with complementary sequences within the PCR amplicon to form an RNA:DNA heteroduplex that can be cleaved by RNase H. When the RNA sequence portion of the probe is cleaved by the RNase, the two parts of the probe, i.e., a donor and an acceptor, dissociate from the target amplicon into a reaction buffer. As the donor and acceptor separate, FRET is reversed and donor emission can be monitored corresponding to the real-time detection of WNV RNA sequences in the sample. Cleavage and dissociation also regenerates a site for further CataCleave™ probe binding on the amplicon. In this way, it is possible for a single amplicon to serve as a target for multiple rounds of probe cleavage until the primer is extended through the CataCleave™ probe binding site.

In certain embodiments, the real-time nucleic acid amplification permits the real-time detection of a single WNV target RNA molecule in less than about 40 PCR amplification cycles.

In other embodiments, the Catacleave™-PCR methodology described herein permits the detection of 100 copies of WNV lineage 1, 10 copies of WNV lineage 1A, 10 copies of WNV lineage 2 and 100 copies of WNV lineage 3.

In certain embodiments, the disclosed methods provide for the detection of one or more WNV strains, including, but not limited to, WNV strains NY99-flamingo 382-99, ArD76104, isolates goose-Hungary/03 and Rabensburg isolate 97-103.

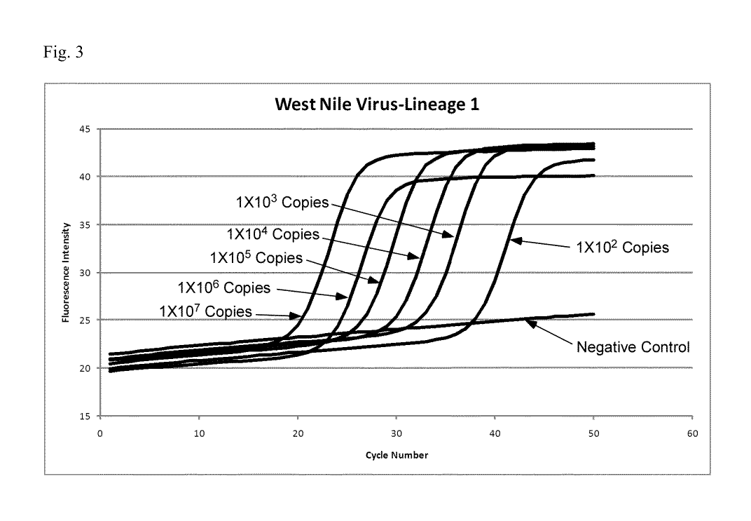

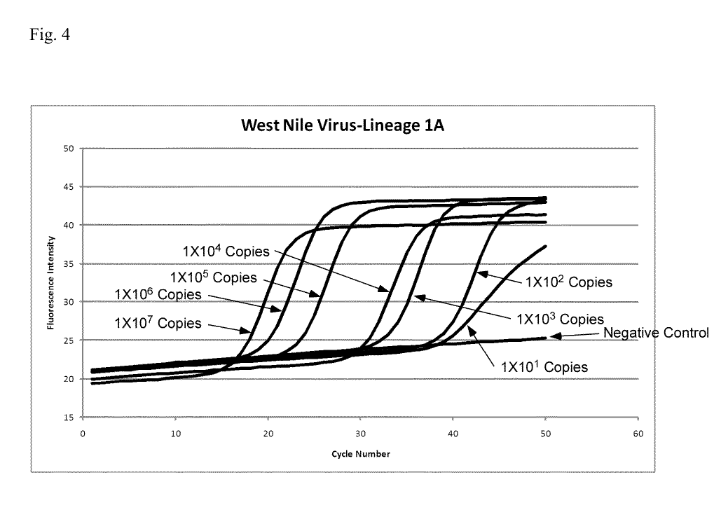

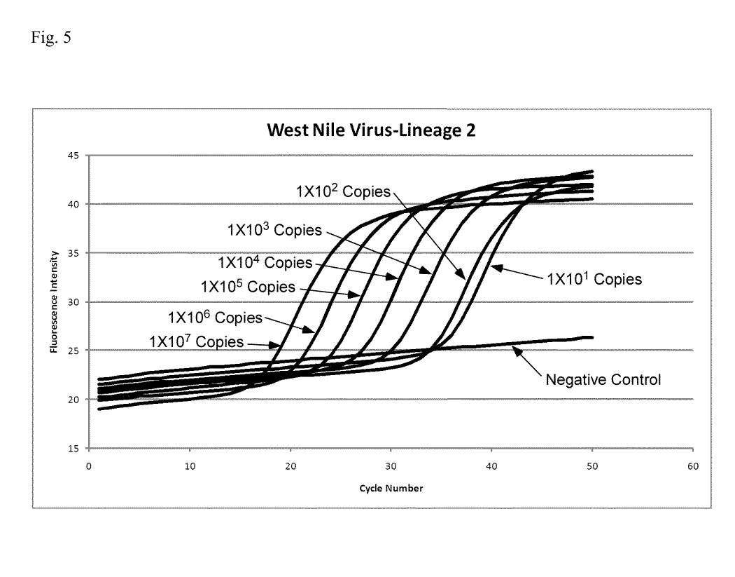

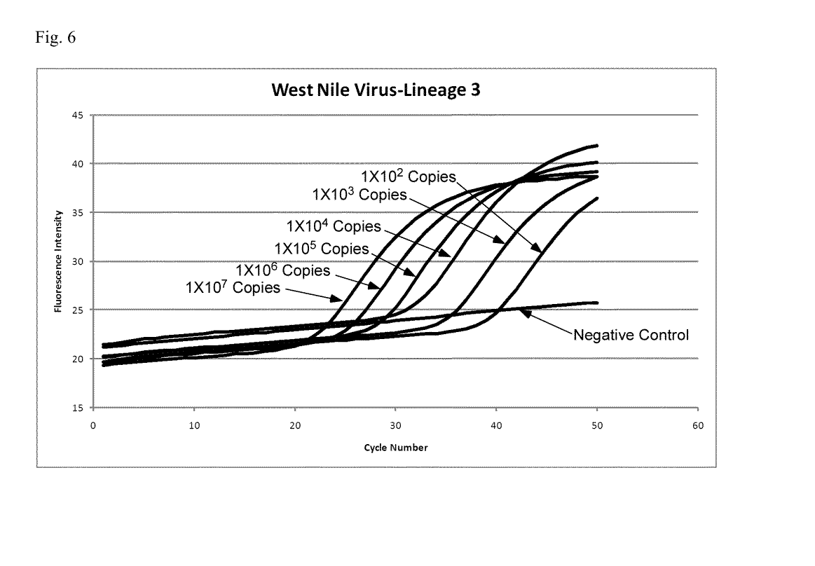

Fluorescence emitted in every cycle of real-time PCR is detected and quantified in real-time using a spectrofluorophotometer, for example, real-time PCR systems 7900, 7500, and 7300 (Applied Biosystems), Mx3000p (Stratagene), Chromo 4 (BioRad), and Roche Lightcycler 480. The real-time PCR device senses the fluorescence marker of the probe of amplified PCR products to show traces as shown in FIGS. 3-6.

### Kits

The disclosure herein also provides for a kit format which comprises a package unit having one or more reagents for the real-time detection of WNV nucleic acid sequences in a sample. The kit may also contain one or more of the following items: buffers, instructions, and positive or negative controls. Kits may include containers of reagents mixed together in suitable proportions for performing the methods described herein. Reagent containers preferably contain reagents in unit quantities that obviate measuring steps when performing the subject methods.

Kits may also contain reagents for real-time PCR including, but not limited to, a reverse transcriptase, thermostable polymerase, RNase H, primers selected to amplify selected WNV nucleic acid sequences and a labeled CataCleave™ oligonucleotide probe that anneals to the real-time PCR product and allow for the detection of the WNV sequences according to the methodology described herein. Kits may comprise reagents for the simultaneous detection of one or more strains of WNV. In another embodiment, the kit reagents further comprised reagents for the extraction of WNV RNA from a biological sample.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material set forth herein is only incorporated to the extent that no conflict arises between that incorporated material and the present disclosure material.

### EXAMPLES

The present invention will be described in further detail with reference to the following examples. These examples are for illustrative purposes only and are not intended to limit the scope of the invention.

### Example 1

### Preparation of Primers and a Probe for the Specific Detection of WNV RNA

To select WNV target nucleic acid sequences for real-time PCR detection, the complete genome sequences of 95 unique WNV virus isolates were aligned and examined for regions of homology. As an example, the sequence alignment for isolate NY99-flamingo 382-99, isolate ArD76104, isolate goose-Hungary/03 and isolate Rabensburg 97-103 are shown in FIG. 7. These four isolates represent represent WNV lineages 1, 1A, 2, and 3. Regions of sequence homology are shaded in black. Locations of the forward and reverse primers and probe are notated and underlined. Homology regions within the nonstructural proteins 1 and 2A genes were selected for further analysis.

A minigene encompassing a selected homology region was then synthesized by Integrated DNA Technologies (Coralville, Iowa) for each WNV lineage and inserted downstream of a T7 RNA polymerase promoter to allow for the generation of WNV RNA target sequences, the detection of which could be tested using different primer and probe sets in real-time reverse transcription PCR reactions. The DNA sequence of each selected homology region is shown below.

Lineage 1:
Accession number AF196835 West Nile virus strain NY99-
flamingo382-99, complete genome
Minigene Sequence 1
(SEQ ID NO: 1)
CGGAAAGTTGATAACAGATTGGTGCTGCAGGAGCTGCACCTTACCACCACTGCGCTACCA
AACTGACAGCGGCTGTTGGTATGGTATGGAGATCAGACCACAGAGACATGATGAAAAGAC
CCTCGTGCAGTCACAAGTGAATGCTTATAATGCTGATATGATTGACCCTTTTCAGTTGGG
CCTTCTGGTCGTGTTCTTGGCCACCCAGGAGGTCCTTCGCAAGAGGTGGACAGCCAAGAT
CAGCATGCCAGCTATACTGATTGCTCTGCTAGTCCTGGTGTTTGGGGGCATTACTTACAC
TGATGTGTTACGCTATGTCATCTTGGTGGGGGCAGCTTTCGCAGAATCTAATTCGGGAGG
AGACGTGGTACAC
Lineage 2: Accession number DQ318019, West Nile virus strain
ArD76104, complete genome.
Minigene Sequence 2
(SEQ ID NO: 2)
CACTGAGAGTGGGAAGCTCATCACAGACTGGTGCTGCAGAAGTTGCACCCTCCCTCCACT
GCGCTTCCAGACTGAGAATGGCTGTTGGTATGGAATGGAAATTCGACCTACGCGGCACGA
CGAAAAGACCCTCGTGCAATCGAGAGTGAATGCATACAACGCCGACATGATTGATCCTTT
TCAGTTGGGCCTTCTGGTCGTGTTCTTGGCTACCCAGGAGGTCCTTCGCAAGAGGTGGAC
GGCCAAGATCAGCATTCCAGCTATCATGCTTGCACTCCTAGTCCTAGTGTTTGGGGGTAT
TACGTACACTGATGTCCTGCGATATGTCATTCTCGTCGGCGCCGCGTTTGCTGAAGCAAA
CTCAGGAGGAGACGTCGTGCACTTGGCACTTATGGCTACA
Lineage 1A: Accession number DQ118127, isolate goose-Hungary/03,
complete genome
Minigene Sequence 1A
(SEQ ID NO: 3)
CACTCGCACCACCACAGAGAGCGGAAAGTTGATAACAGATTGGTGCTGCAGGAGCTGCAC
CTTACCACCACTGCGCTACCAAACTGACAGCGGCTGTTGGTATGGTATGGAGATCAGACC
ACAGAGACATGATGAAAAGACCCTCGTGCAGTCACAAGTGAATGCTTACAATGCTGATAT
GATTGACCCTTTTCAGTTGGGCCTTCTGGTCGTGTTCTTGGCCACCCAGGAGGTCCTTCG
CAAGAGGTGGACAGCCAAGATCAGCATGCCAGCTATACTGATTGCTCTGTTAGTCCTGGT
GTTTGGGGGCATTACTTACACTGATGTGTTACGCTATGTCATTTTGGTGGGGGCAGCTTT
TGCAGAATCTAATTCGGGAGGAGACGTGGTACACTTGGCG
Lineage 3:
Accession number AY765264, strain Rabensburg isolate 97-103,
complete genome.
Minigene Sequence 3
(SEQ ID NO: 4)
CGCGCGCACCACCACAGAGAGTGGGAAGTTGATCACGGATTGGTGCTGCAGAAGCTGCAC
GCTCCCCCCACTACGGTATCAAACTGATAGTGGATGTTGGTATGGAATGGAAATCAGACC
TTTGAAGCATGATGAGAAGACGTTGGTTCAATCTAGGGTGAGCGCCTACAAATCTGATAT
GATTGATCCTTTTCAGCTGGGCCTTCTGGTAGTGTTCTTGGCCACCCAGGAGGTCCTCCG
CAAGAGGTGGACAGCCAAGATCAGCATTCCTGCTATTCTGGTCGCTCTTGCAGTCCTAGT
GCTTGGGGGCATCACTTACACTGATGTTCTGAGATACATCATTCTTGTGGGTGCGGCCTT
TATGGAAGCCAACTCAGGTGGAGATGTGGTGCATCTTGCT

The following primers were selected, according the methods cited herein, for the real time PCR detection of lineage-specific WNV target nucleic acid sequences:

F1-3 lineage 1:
(SEQ ID NO: 5)
TGT TGG TAT GGT ATG GAG AT
F1-3 lineage 2:
(SEQ ID NO: 6)
TGT TGG TAT GGA ATG GAA AT
R3-6 lineage 1:
(SEQ ID NO: 7)
AGT GTA AGT AAT GCC CCC
R3-6 lineage 2:
(SEQ ID NO: 8)
AGT GTA CGT AAT ACC CCC

Sequences in bold and underlined denote changes from lineage 1 primers.

CataCleave™ Probe:

WNV4 probe:
(SEQ ID NO: 9)
5′FAM/ATG ATT GAC CCrU rUrUrU CAG TTG GGC CTT/
3′IABlk FQ

Lowercase ‘r’ denotes ribonucleotides

5′FAM: 5′ 6 -carboxy fluorescein

3′ IABlk FQ: 3′ Iowa Black FQ Quencher

### Example 2

### Template RNA Preparation

RNA was synthesized from each minigene described above using the HiScribe T7 In Vitro Transcription Kit (New England Biolabs, catalog #E2030S) according to the manufacturer's instructions. Plasmid DNA was then digested with DNase I. Unincorporated ribonucleotides and deoxyribonucleotides were removed on DNA grade G-25 spin columns (G.E.). RNA concentrations were then calculated by A (260 nm) absorbance where 1 OD (260 nm)=40 μg/ml and the RNA was diluted in 5 mM Tris-HCl, pH 8.0.

### Example 3

### Real-Time Reverse Transcription PCR

RNA representing the four lineages was serially diluted from 5×10⁶/μl to 5 copies/μl in 5 mM Tris-HCl, pH 8.0.

Each RT-PCR reaction contained:

| Component | Volume (μl) |
|---|---|
| 5X reaction buffer | 5 |
| 10 mM dA, dC, dG + 5 mM dT mix | 0.5 |
| 50X four primer: probe mix | 0.5 |
| Bacillus heat-labile UDG (57 ng/ul) | 0.4 |
| Pyrococcus furiosis hot-start RNase HII | 0.5 |
| Invitrogen Platinum Taq (5 U/ul) | 0.5 |
| Invitrogen Superscript III reverse transcriptase (200 U/ul) | 0.5 |
| Diluted WNV RNA | 2.0 |
| H₂O | 15.1 |
| TOTAL | 25 |

The 50× four primer: probe mix contained 12 μM F1-3 lineage 1, F1-3 lineage 2, R3-6, lineage 1, R3-6 lineage 2 primers (SEQ ID NOs: 5-8 respectively) and 10 μM WNV4 probe (SEQ ID NO: 9).

Real-time reactions were performed in a Roche LightCycler LC480 II using the following protocol:

50° C. for 20 minutes (reverse transcription of WNV RNA),

95° C. for 5 minutes to inactivate the reverse transcriptase and UDG activities and activate Pfu RNase HII and Taq DNA polymerase,

50 cycles of 95° C. for 10 seconds, 55° C. for 10 seconds, 65° C. for 30 seconds

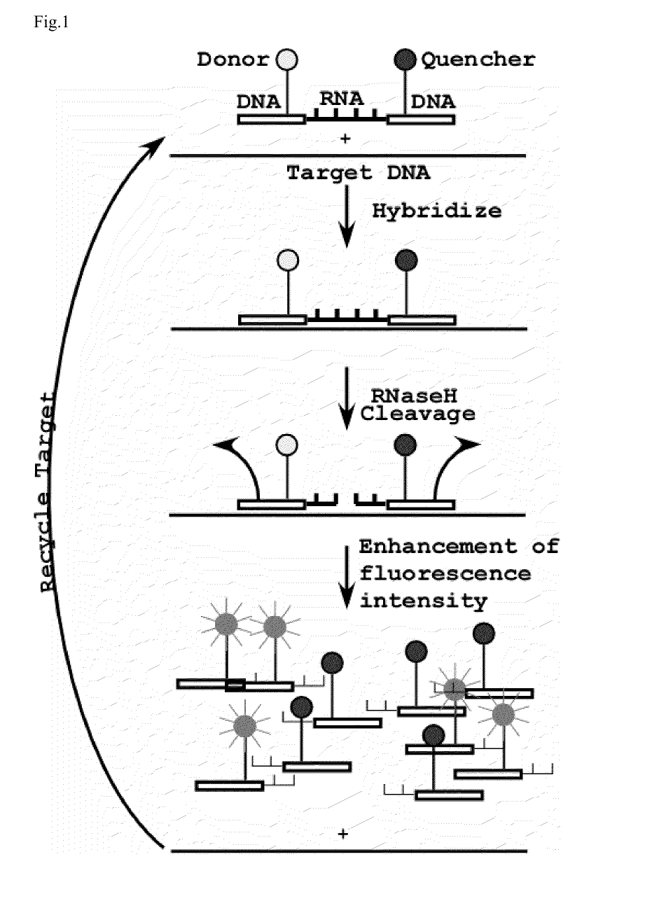

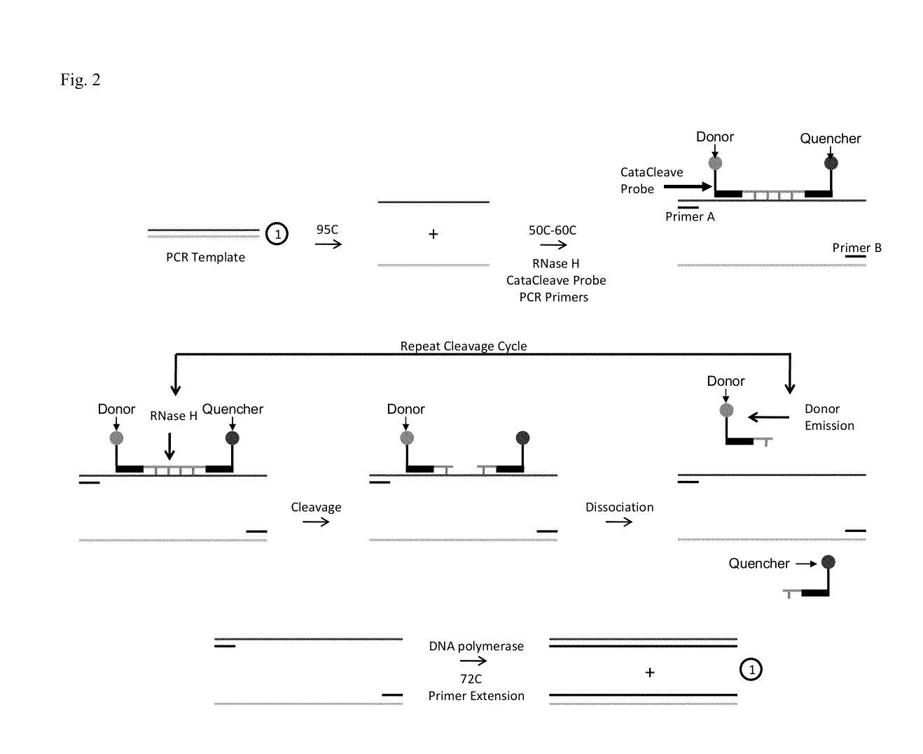

Results are shown in FIGS. 1-4.

The results show that the limit of detection for WNV lineages was as follows:

| | |
|---|---|
| WNV Lineage 1 | 100 copies |
| WNV lineage 1A | 10 copies |
| WNV lineage 2 | 10 copies |
| WNV lineage 3 | 100 copies. |

What is claimed is:

1. A method for the real-time detection of West Nile Virus (WNV) in a sample, comprising the steps of:
a) providing a sample to be tested for the presence of a WNV target nucleic acid sequence;
b) providing a pair of forward and reverse amplification primers, wherein the primer pair anneals to a WNV homology region of SEQ ID NO: 1, 2, 3 or 4 comprising the WNV target nucleic acid sequence;
c) providing a probe comprising a detectable label and DNA and RNA nucleic acid sequences, wherein the probe's RNA nucleic acid sequences are entirely complementary to a selected region of the target WNV cDNA and the probe's DNA nucleic acid sequences are substantially complementary to WNV cDNA sequences adjacent to the selected region of the target DNA sequence;
d) reverse transcribing the WNV target RNA in the presence of a reverse transcriptase activity and the reverse amplification primer to produce a target WNV cDNA sequence;
e) amplifying an PCR fragment between the forward and reverse amplification primers in the presence of the WNV target cDNA sequence, an amplifying polymerase activity, an amplification buffer; an RNAse H activity and the probe under conditions where the RNA sequences within the probe can form a RNA:DNA heteroduplex with complimentary sequences in the PCR fragment; and
detecting a real-time increase in the emission of a signal from the label on the probe, wherein the increase in signal indicates the presence of the WNV target nucleic acid sequences in the sample.

2. A method for the real-time detection of West Nile Virus (WNV) in a sample, comprising the steps of:
a) providing a sample to be tested for the presence of a WNV target nucleic acid sequence;
b) providing a pair of forward and reverse amplification primers that can anneal to the WNV target nucleic acid sequence, wherein the forward amplification primer can be the primer of SEQ ID NO: 5 or 6 and the reverse amplification primer can be the primer of SEQ ID NO: 7 or 8;
c) providing a probe comprising a detectable label and DNA and RNA nucleic acid sequences, wherein the probe's RNA nucleic acid sequences are entirely complementary to a selected region of the target WNV cDNA and the probe's DNA nucleic acid sequences are substantially complementary to WNV cDNA sequences adjacent to the selected region of the target DNA sequence;
d) reverse transcribing the WNV target RNA in the presence of a reverse transcriptase activity and the reverse amplification primer to produce a target WNV cDNA sequence;
e) amplifying an PCR fragment between the forward and reverse amplification primers in the presence of the WNV target cDNA sequence, an amplifying polymerase activity, an amplification buffer; an RNAse H activity and the probe under conditions where the RNA sequences within the probe can form a RNA:DNA heteroduplex with complimentary sequences in the PCR fragment; and
f) detecting a real-time increase in the emission of a signal from the label on the probe, wherein the increase in signal indicates the presence of the WNV target nucleic acid sequences in the sample.

3. The method of claim 2, wherein the real-time increase in the emission of the signal from the label on the probe results from the RNAse H cleavage of the probe's RNA sequences in the RNA:DNA heteroduplex.

4. The method of claim 2, wherein the DNA and RNA sequences of the probe are covalently linked.

5. The method of claim 2, wherein the detectable label on the probe is a fluorescent label.

6. The method of claim 2, wherein the probe is labeled with a FRET pair.

7. The method of claim 2, wherein the PCR fragment or probe is linked to a solid support.

8. The method of claim 2, wherein the RNAse H activity is the activity of a thermostable RNAse H.

9. The method of claim 2, wherein the RNAse H activity is a hot start RNAse H activity.

10. The method of claim 2, wherein the probe comprises the sequence of SEQ ID NO: 9.

11. The method of claim 2, wherein the real-time increase in the emission of a signal from the label on the probe can detect 100 copies of WNV lineage 1, 10 copies of WNV lineage 1A, 10 copies of WNV lineage 2 and 100 copies of WNV lineage 3.

12. A kit for the real-time detection of West Nile Virus (WNV) in a sample, comprising:
a) a reverse transcriptase activity for the reverse transcription of a target West Nile Virus (WNV) RNA sequence to produce a target cDNA sequence;
b) a pair of forward and reverse amplification primers that can anneal to the WNV target nucleic acid sequence, wherein the forward amplification primer can be the primer of SEQ ID NO: 5 or 6 and the reverse amplification primer can be the primer of SEQ ID NO: 7 or 8;
c) an amplifying activity for the PCR amplification of the target WNV cDNA sequence between the pair of amplification primers to produce a West Nile Virus (WNV) PCR fragment;
d) a probe comprising a detectable label and DNA and RNA nucleic acid sequences, wherein the probe's RNA nucleic acid sequences are entirely complementary to a selected region of the target WNV cDNA and the probe's DNA nucleic acid sequences are substantially complementary to WNV cDNA sequences adjacent to the selected region of the target DNA sequence, and
e) an RNAse H activity.

13. The kit of claim 12, further comprising positive, internal, and negative controls.

14. The kit of claim 12, wherein the detectable label on the probe is a fluorescent label.

15. The kit of claim 12, wherein the probe is labeled with a FRET pair.

16. The kit of claim 12, wherein the probe or PCR fragment is linked to a solid support.

17. The kit of claim 12, wherein the kit further comprises an amplifying polymerase activity.

18. The kit of claim 12, wherein the RNAse H activity is the activity of a thermostable RNAse H.

19. The kit of claim 12, wherein the RNAse H activity is a hot start RNAse H activity.

20. The kit of claim 12, wherein the probe comprises the sequence of SEQ ID NO: 9.
